(19) United States
(12) Patent Application Publication
(10) Pub. No.: US 2012/0034273 A1
(43) Pub. Date: Feb. 9, 2012

(54) EXTRUDATE HAVING SPICULAR ACTIVE SUBSTANCES

(75) Inventors: Venkata-Rangarao Kanikanti, Leverkusen (DE); Hans-Juergen Hamann, Dormagen (DE); Peter Kleinebudde, Dusseldorf (DE); Rieke Witzleb, Berlin (DE)

(73) Assignee: Bayer Animal Health GmbH, Leverkusen, DE (US)

(21) Appl. No.: 13/132,872

(22) PCT Filed: Nov. 24, 2009
(86) PCT No.: PCT/EP2009/008341

§ 371 Date: Oct. 20, 2011

(30) Foreign Application Priority Data
Dec. 5, 2008 (DE) 102008060472.0

(51) Int. Cl. A61K 9/00; A61K 9/16

(52) U.S. Cl. 424/400; 428/402

(57) ABSTRACT

The invention relates to extrudates containing at least one pharmaceutically active substance in the form of needles, wherein the ratio of the particle size of the needle-shaped pharmaceutically active substance to the strand diameter is at least 1:15, and the use of these extrudates for the production of medicaments.

The invention relates to extrudates containing at least one pharmaceutically active substance in the form of needles, wherein the ratio of the particle size of the needle-shaped pharmaceutically active substance to the strand diameter is at least 1:15, and the use of these extrudates for the production of medicaments.

Masking of the taste of bitter drug substances is important for improving compliance with human medicaments, not least for pediatric formulations, but also in veterinary medicaments. The simplest type of taste masking is the addition of aromas, which can be a problem with very bitter and very water-soluble substances (Bienz, 1996). Taste masking by processing, of an active substance into granules with a hydrophobic carrier has also been described (Kalbe and Hopkins, 1998). Another possibility is the coating of drug forms. Examples of materials used for this are Eudragit E (Cerea et al., 2004; Lovrecich et al., 1996; Ohta, and Buckton, 2004; Petereit and Weisbrod, 1999), shellac (Pearnchob et al., 2003b; Pearnchob et al., 2003a) and cellulose derivatives (Al-Omran et al., 2002; Li et al., 2002; Shirai et. al., 1993). The disadvantage of Eudragit E is that the taste masking is based on an ionic interaction between a cationic additive and anionic active substances. The disadvantage of shellac is that it is a natural polymer, whose composition may vary. Apart from this, the coating of drug forms is an additional cost- and time-intensive processing step. In addition, solid dispersions of quinolone- or naphthyridonecarboxylic acids in an insoluble shellac matrix have been described (Cabrera, 2002).

Ion exchange resins and inclusion complexes are also used for taste masking. The usability of the ion exchange resins is limited by the fact that the drug substance must have ionic properties (Chun and Choi, 2004; Lu et al., 1991; Prompruk et al., 2005). Inclusion complexes can only be loaded with small quantities of drug substances (Sohi et al., 2004).

Lipid bases can also be used for taste masking. Monolithic drug forms based oh hard fat, which also contain lecithin and sweeteners for taste masking; have been described (Suzuki et al., 2003; Suzuki et al., 2004). The disadvantage here is that during the production process the lipids must be completely melted, which can lead to physical instability. Furthermore, hard fat, glycerol distearate and stearic acid have been used as lipophilic binders in cold extrusion, with the use of Eudragit E as a coating also being necessary here in order to achieve taste masking (Breitkreutz et al., 2003). The extrusion of fats below their melting point for the production of drug, forms has also been described, albeit not for the purpose of taste masking (Reitz and Kleinebudde, 2007; Windbergs et al., 2008).

Taste masked formulations with gyrase inhibitors of the quinolone type have been obtained by mixing the active substance with higher fatty acids and if necessary other additives, heating, and granulating or pulverizing after cooling (Ahrens et al.; 1998). Furthermore, pellets based on waxes have been produced (Adeyeye and Price, 1991; Adeyeye and Price, 1994; Zhou et al., 1996; Zhou et al., 1998). The studies showed that the release of the substances depends on the melting point of the wax used and the concentration thereof in the pellet. The higher the melting point and the content of wax were the slower the release. A further possibility for taste masking is described by Kim and Choi (2004), who prepared a core of cocoa butter or hard fat and the active substance and provided this with a coating of sodium alginate or carrageenan. In the process, however, the fat was completely melted, which was disadvantageous for stability reasons, moreover the production step for the coating was an additional process step.

In addition, Compritol® 888 ATO has been described as a matrix-forming component. The pellets consisted of melted Compritol®, the active substance and a polysaccharide coating (Mirghani et al., 2000). In another study, matrix tablets, were pressed either directly from a powder mixture or a pulverized solid dispersion. The tablets from the pulverized solid dispersion exhibited better taste masking, however for the production of these the Compritol® had to be completely melted (Li et al., 2006). Barthelemy used Compritol® for the coating of theophylline pellets and granules. Here also, the fat was melted completely (Barthelemy et al., 1999).

The use of phospholipids is another possibility for the masking of bitter taste, but not of other taste types (Katsuragi et al., 1997; Takagi et al., 2001). In addition, the addition of phospholipids affects the crystallinity of the lipids, which can lead to instability (Schubert, 2005). Taste masking of powders is possible by depositing small additive particles onto large substance particles (Barra et al., 1999).

Animal feed into which active substances had been incorporated by extrusion has also been described (Huber et al., 2003). By melt extrusion of a basic drug substance and a methacrylate polymer. Petereit et al. obtained taste masked extrudates which were then milled into granules or powder (Petereit et al., 2003) and a rapidly disintegrating drug form by mixing of the two components with a medium to long-chain fatty acid in the melt. After solidification, the product was milled and embedded into a water-soluble matrix. (Petereit et al., 2004). Medicaments with controlled release, which contained the active substance in a lipid matrix of behenate esters and a hydrophobic diluent have been studied (Nabil et al., 1998). Thombre describes a formulation for pets in multiparticulate form with a taste masking additive (Thombre, 2004).

In the pending application “Extrudates with improved taste masking” (German patent application File No. 102007026550.8, see also corresponding PCT application No. PCT/EP2008/004218) pharmaceutically usable extrudates with a strand diameter of 0.5 mm and less were described. These extrudates are suitable for the taste masking of drug substances.

However, in the extrusion of mixtures which contain active substances in needle form, unexpected difficulties arise: if the crystal form of the drug substances used is, needle-shaped, no stable and reproducible production processes can be carried out, even the length of the needles is markedly smaller than the strand diameter. In principle, processing difficulties with needle-shaped drug substances were well known with other production technologies. It has been shown in various studies that the properties of powders and the resulting tablets depend on the particle form of the substances used (Alderborn and Nyström, Wong and Pilpel, 1990). For example, paracetamol in the needle-shaped crystal habit can be compressed into tablets much less well than in other crystal forms, which manifested itself in capping tablets and poor powder flowability (Wang & Zhang, 1995). In the needle-shaped crystal form, ibuprofen also displayed very poor flowability, cohesive and adhesive properties, a high energy input was necessary for compacting and tabletting, and the resulting, tablets were mechanically unstable. By agglomeration or recrystallization into isometric crystal forms, the tabletting properties of ibuprofen could be markedly improved (Jbilou et al., 1999; Rasenack and Müller, 2002).

However, as regards the extrusion process it was known from glass processing that needle-shaped particles arrange themselves in the direction of extrusion. In this case, an alignment was desired, in order to achieve anisotropic properties in the glasses produced (Moisescu et al., 1999). In comparison with isometric particles, the viscosity of the softened glass mass during the melt extrusion was markedly higher if it contained needle-shaped particles (Yue et al., 1999).

The person skilled in the art would have expected rib problem so long as the particle size is smaller by a considerable factor (e.g. 5) than the strand diameter, in particular since he could certainly assume that the particles would align themselves parallel, favourably, for extrusion.

Surprisingly however, problems arise in the extrusion of mixtures containing needle-shaped active substances: the problems in the production process manifest themselves in the form of build-ups before the nozzle plate, so that nozzle apertures are blocked and as a result of this the pressure before the extruder nozzle plate rises. Apart from this, the powder mixtures flow so poorly that it is almost impossible to maintain a constant feed rate at high processing speeds.

In order to arrive at a satisfactory extrusion process for needle-shaped active substances, various approaches are, possible: modifications in the formula, e.g. variation of the lipid base, addition of further additives or experiments with various active substance loadings do not produce sufficient improvements. Furthermore, nozzle plates with stepwise widening of the nozzle channel in the process direction can be used, however in the present case this under some circumstances leads to a deterioration in the process uniformity. Even extrusion through nozzle plates with especially smooth surfaces in the aperture drillings does not result in any significant change. As a further equipment variation, different screw configurations can be used, but this also does not result in any improvement. Further, the process parameters of temperature, feed rate and screw revolution rate can be varied. Extrusion temperatures of 20° C. below the melting range of the lipid used up to within the melting range are possible. At too low a temperature, the nozzles block immediately and the pressure rises very rapidly, and at too high a temperature the lipids melt completely and leave the nozzles as a soil paste.

However, it is found that with larger nozzle diameters certain improvements are achieved. Since none of the other modifications decisively improves the process, the active substance powder is milled (e.g. in an air jet mill); the needle-shaped crystal structures are pulverized by the milling. It is found that at sufficiently small particle size the extrudates can be produced without any problems. The extrusion process with sufficiently finely milled active substance as a rule proceeds smoothly and reproducibly at constant pressure, even with high drug substance loading (e.g. 50% or even up to 80%) and a nozzle diameter of for example 0.3 mm or even only 0.2 mm.

The invention thus relates to:
extrudates containing at least one pharmaceutically active substance in the form of needles, characterized in that the ratio of the particle size of the needle-shaped pharmaceutically active substance to the strand diameter is at leak 1:15.
the use of the aforesaid extrudates for the production of medicaments.

The ratio of the particle size of the needle-shaped pharmaceutically active substances to the strand diameter is usually at least 1:15, preferably at least 1:20, particularly preferably at least 1:25, quite particularly preferably at least 1:50, in particular at least 1:100.

In case of doubt, particle size should here be understood to mean the d(0.9) value determined by laser diffractometry. In the sense of this invention, d(0.9) is understood to mean a volume-based particle size distribution in which 90% of all particles have a dimension (diameter) less than or equal to this value (occasionally the terms d(90) or d(v,90) are also used for this, the latter in order to make it clear that this is a volume-based particle size distribution). The terms d(0.5), d(0.1) and the like should be understood correspondingly. The particle sizes stated here were determined by the laser diffraction method with the Malvern Mastersizer 2000 (dispersion unit Hydro 2000G) and the Fraunhofer diffraction evaluation mode, since the refractive indices of the active: substance particles are not known. For this, a suitable quantity of the sample solution was predispersed with 2-3 ml of a dispersion medium (e.g. a 0.1% aqueous dioctyl sodium sulphosuccinate solution for praziquantel or ethanol for mesalazine) with stirring. The dispersion was then fed into the dispersion unit of the instrument with stirring (300 rpm) and pumping, (900 rpm) and the measurement made. The evaluation software outputted the particle size as d(0.9) values (or d(0.5) values or the like).

Active substance particles which are too soluble in common solvents (e.g. caffeine) are dry dispersed with a suitable unit (e.g. Scirocco 2000 dry powder feeder) by means of an air flow at 0.5 bar air pressure.

The strand diameter of the extrudate according to the invention is preferably at most 0.5 mm, particularly preferably at most 0.3 mm. Normally extrudates beyond a diameter of 0.1 mm, preferably beyond 0.2 mm, can be used. With non-cylindrical extrudates, the maximum edge length or ellipse length is at most 0.5 mm, preferably at most 0.3 mm.

The extrudates contain a base suitable for extrusion made of a thermoplastically deformable material or a mixture of several thermoplastically deformable materials and if necessary further pharmaceutically acceptable auxiliary agents and additives.

The base consists of thermoplastically deformable materials such as polymers, for example polyacrylates or cellulose derivatives, lipids, for example acylglycerides, surfactants, for example glycerol monostearate or sodium stearate, macrogels, for example polyethylene glycol 6000, sugars or sugar alcohols, for example mannitol or xylitol. Preferably a lipid base is used. As the lipid base for example fatty bases, in particular glycerol esters, are suitable, and these are preferably esters with C₁₂-C₂₄ fatty acids. As glycerol esters, glycerol diesters, such as for example glycerol dibehenate, glycerol triesters, such as for example glycerol trilaurate, glycerol trimyristate, glycerol tripalmitate or glycerol tristearate, and mixtures of glycerol mono-, di- and triesters, such as for example glycerol palmitostearate, may be mentioned. Triglycerides based on coconut butter, palm oil and/or palm nut oil (such as for example the hard fats obtainable in commerce under the name Witocan®) may also be mentioned. Mono- or diglycerides of citric and/or lactic acid are also usable.

Further, waxes, in particular those with 30 to 60 carbon atoms, such as cetyl palmitate, may be mentioned.

Such lipids are for example commercially available under the names Precirol®, Compritol® and Dynasan®.

A particularly preferred example from the glycerol diester series is glycerol dibehenate (e.g. Compritol® 888 ATO, which mainly contains glycerol dibehenate but also glycerol monobehenate and glycerol tribehenate). Particularly preferred examples from the glycerol triester series are glycerol trimyristate (e.g. Dynasan® 114), glycerol tripalmitate (e.g. Dynasan® 116) and glycerol tristearate (e.g. Dynasan® 118).

Preferably the fatty bases are in powder form. Many lipids are polymorphous and can under some circumstances form metastable forms in the event of temperature and pressure changes. On storage under some circumstances, conversions of the modifications can occur, and more stable modifications be formed. According to descriptions in the literature [Reitz and Kleinebudde, 2007; Windbergs et al., 2008] glycerol triesters (known for example as Dynasan®), in particular glycerol trimyristate (Dynasan® 114®) or also glycerol tripalmitate (e.g. Dynasan® 116) or glycerol tristearate (e.g. Dynasan® 118) are comparatively stable against such changes and are therefore particularly suitable as the lipid base for medicaments.

In particular the substances used as fatty bases are often sold as mixtures, e.g. of mono-, di- and/or triglycerides. Homogeneous fatty bases, which essentially consist of only one component, are preferable to these. Formulations produced with these additives are characterized by good storage stability.

The quantity of base (made of thermoplastically deformable; materials) used depends on the quantity of the other substances contained in the extrudate. Normally 15 to 99% [w/w], preferably 20 to 99% [w/w], particularly preferably 25 to 80% [w/w], quite particularly preferably 30 to 70% [w/w] is used.

The bases used, in particular the lipid bases, as a rule have a melting range whose lower limit usually is at least 50° C., preferably at least 60° C.

The extrudates according to the invention can if necessary contain one or more further auxiliary agents and additives. Possible as these are flow regulators, preferably colloidal silicon dioxide at a concentration of 0.2 to 2% [w/w], lubricants, preferably magnesium stearate or calcium dibehenate at a concentration of 0.2 to 5% [w/w], and surfactants; preferably lecithin, at a concentration of 0.5 to 10% [w/w]. Further, antioxidants can be used, and for example butylhydroxyanisole (BHA) or butylhydroxytoluene (BHT), which are used, in normal quantities, as a rule 9.91 to 0.5% [w/w], preferably 0.05 to 0.2% [w/w] are suitable. The release of active substance can for example be controlled by addition of so-called pore forming materials. Examples of these are sugars, in particular lactose, polyols, in particular mannitol or polyethylene glycols (PEG), preferably PEG 1500 to 10 000, particularly preferably PEG 1500 to 6000, e.g. PEG 1500 (Macrogol 1500). The pore forming materials are used at a concentration of 5 to 40% [w/w], preferably at a concentration of 5 to 20% [w/w]. Another possibility for influencing the release of active substance is the addition of disintegration aids. In addition, so-called super disintegrators such as crospovidone, croscarmellose sodium or crosslinked sodium carboxymethylstarch can be used. The super disintegrators are used at a concentration of 1 to 15% [w/w], preferably at a concentration of 3 to 10% [w/w]. Alternatively to this, substances, can be used which are soluble in acids and/or evolve carbon dioxide such as magnesium carbonate or calcium carbonate. The carbon dioxide-releasing substances are used at a concentration of 5 to 15% [w/w], preferably at a concentration of 5 to 10% [w/w].

Further, the extrudates according to the invention can contain antistatic agents. This is particularly to be recommended if electrostatic charges affect the extrusion. Electrostatic charges can result in blockage of the nozzle apertures, which can be prevented by addition of antistatic agents. As antistatic agents, PEG can preferably be used, particular possibilities being PEG 1500 to 6000. The PEG should preferably be in powder form and melt during the extrusion, in order to exert an antistatic effect. Hence with the addition of PEG as an antistatic agent, the melting temperature of the PEG should be sufficiently low that in the extrusion the PEG does melt, but not the fatty base used. In practice, static charges do not arise with all bases. They are above all observed with glycerol fatty acid triesters with fatty acids, such as for example glycerol trimyristate, glycerol tripalmitate or glycerol tristearate. Hence with the use of such bases it is recommended to add an antistatic agent to the mixture to be extruded, in order to ensure problem-free extrusion. The antistatic agents are used at a concentration of at least 5% [w/w], preferably of at least 10% [w/w]. Usually not more than 30% [w/w] is used.

As pharmaceutically active substances, drug active substances can be used, and in particular those whose unpleasant taste has to be masked.

According to the invention, active substances which are needle-shaped are used. As a general rule, these are needle-shaped crystals.

Here, “needle” or “needle-shaped” should be understood to mean particles whose length is markedly greater than their diameter, in which the length/diameter ratio is at least greater than 3:1, preferably greater than 5:1, particularly preferably greater than 10:1, quite particularly preferably greater than 20:1. Since the needles are as a rule not round, incase of doubt, “diameter” should be understood to mean the greatest dimension perpendicular to the length.

Essentially there is no great restriction in the choice of the needle-shaped active substance, since it is not necessary to melt the active substance. Because of the taste masking, action of the extrudate, they are preferably suitable, for unpleasant—e.g. bitter—tasting active substances.

The active substances can for example belong to the following groups: antibiotics; drugs against parasitic protozoa, anthelmintics, metabolism-stimulating agents and inflammation inhibiting substances.

Of course, the term active substance also includes needle-shaped salts and solvates.

As a specific example of an active substance, mesalazine may be mentioned. Mesalazine is an inflammation-inhibiting drug substance which is used against chronic inflammatory intestinal diseases. It is very poorly soluble in water, has a melting point of 280° C. and a needle-shaped crystal form.

A further example is crystalline caffeine.

As a preferred specific example of a needle-shaped active substance, praziquantel, a long-known anthelmintic which is active against tapeworms and schistosomes, may be, mentioned. It is poorly soluble in water, and has a melting point of 139° C. and a needle-shaped crystal form. A grade milled in a pinned disc mill with a particle size of 25 μm, expressed as d(0.9), measured by laser diffractometry (FIG. 1), is normally used.

By embedding in a lipophilic matrix; depending on the nature of the active substance used, a delayed release and hence a retard effect can be achieved.

The quantity of active substance used in the extrudates depends on the potency of action and the desired dosage. It is found that even extrudates with high active substance concentrations of up to 80% [w/w], preferably up to 70% [w/w], particularly preferably up to 60% [w/w] can be produced. Examples of normal concentration ranges are 1 to 80% [w/w], preferably 5 to 70% [w/w] and particularly preferably 30 to 60% [w/w].

The extrudates according to the invention are produced by mixing the starting materials (the pharmaceutically active substance(s), the base and if necessary auxiliary agents and additives) and then extruding. The extrusions are preferably, performed at a temperature which does not lead to complete melting of the thermoplastically deformable materials, and in fact normally at a temperature in the region of room temperature, preferably of 40° C., up to below the melting range of the thermoplastically deformable materials. In practice, this is normally based on the melting range, stated for the base in question. As a rule, the extrusion temperature is set not lower than 20° C., preferably 15° C., particularly preferably 10° C., below the lower limit of the melting range of the base, in particular the fatty base. Normally, the extrusion temperature is set not higher than the lower limit of the melting range of the base, preferably 1° C. lower, particularly preferably 5° C. lower. The aim is to avoid the extrusion of a soil paste. The extrusion process should be carried out at as constant a material temperature as possible. For this purpose, screw extruders, in particular twin screw extruders, are especially suitable. The extruded strand preferably has a circular, cross-section and a diameter as stated above. The extruded strand can be comminuted directly on extrusion with a knife or in a separate step by gentle grinding in a normal mill, e.g. in a centrifugal mill. The grain size of the product obtained depends on the diameter of the nozzle used, and the comminuted strands have at most a length which corresponds to three times the strand diameter. Typical grain sizes are for example 300 to 500 μm or even 200 to 500 μm in case of a smaller nozzle diameter. According to a preferred embodiment, the milled product, can also be screened. In this way, the fines fraction can be removed.

The statement occasionally used here, that the extrudates are extruded below their melting point, should be understood to mean that, as stated above, the extrudates are extruded at a temperature at which the thermoplastic base used does not yet completely melt. Often other components, for example the active substances, have a higher melting point. Such extrudates are suitable for the taste masking of unpleasant tasting components.

After gentle grinding, the extrudates according to the invention can if necessary be further processed into suitable drug forms. The addition of further additives is occasionally necessary for the further processing. The drug forms preferred according to the invention are tablets, which can if necessary have shapes suited to the desired application. Other drug forms which are possible are pastes, suspensions, sachets, capsules, etc.

The extrudates or medicaments according to the invention are generally suitable for use in man and in animals. They are preferably used in animal husbandry and animal bleeding in agricultural, breeding, zoo, laboratory and experimental animals and “hobby animals”, and in particular in mammals.

Agricultural and breeding animals include mammals such as for example cattle, horses, sheep, pigs, goats, camels, water buffaloes, donkeys, rabbits, fallow deer, reindeer, fur animals such as for example mink, chinchilla, raccoon, and birds such as for example chickens, geese, turkeys, ducks, pigeons and ostrich types. Examples of preferred agricultural animals are cattle, sheep, pigs and chickens.

Laboratory and experimental animals include dogs, cats; rabbits and rodents such as mice, rats, guinea pigs and hamsters.

Pets include dogs, cats, horses, rabbits, rodents such as hamsters, guinea pigs, mice, and also reptiles, amphibians and birds for keeping at home and in zoos.

The extrudates are normally used enterally; in particular orally, directly or in the form of suitable, preparations (drug forms).

Enteral use takes place for example orally in the form of granules, tablets, capsules, pastes, suspensions or medicated animal feed. One portion for oral administration is so-called top dressing, this being a powder, granules or a paste which is placed on the animal feed and ingested with the feed.

Suitable preparations are:

solid preparations such as for example granules, pellets, tablets, boluses and active substance-containing moulded bodies.

For the production of solid preparations, the comminuted extrudates are mixed with suitable carriers if necessary with the addition of additives, and made into the desired form.

As carriers, all physiologically compatible solid inert substances may be mentioned. Inorganic and organic substances are used as such. Examples of inorganic substances are common salt, carbonates such as calcium carbonate, hydrogen carbonates, aluminium oxides, silicic acids, aluminas, precipitated or colloidal silicon dioxide and phosphates.

Examples of organic substances are sugar, cellulose, foodstuffs and animal feeds such as powdered milk, animal meal, cereal flour and grist, and starches.

Additives are preservatives, antioxidants and colorants. Suitable additives and the necessary quantities to be added are essentially known to the person skilled in the art. As a preservative, for example sorbic acid may be mentioned. As antioxidants, for example butylhydroxyanisole (BHA) or butylhydroxytoluene (BHT) are suitable. Possible colorants are organic and inorganic colorants or pigments suitable for pharmaceutical purposes such as for example iron oxide.

Further suitable additives are lubricants and parting agents such as for example magnesium stearate, stearic acid, talc, bentonite, disintegration-promoting substances such as starch, or cross-linked polyvinylpyrrolidone, binders such as for example starch, gelatine or linear polyvinylpyrrolidone and dry binders such as microcrystalline cellulose.

As further additives, oils such as plant oils (e.g. olive oil, soya oil, sunflower oil) or oils of animal origin such as for example fish oil can be used Normal quantities are 0.5 to 0% [w/w], preferably 0.5 to 10% [w/w], particularly preferably 1 to 2% [w/w].

Suspensions can be used orally. They are produced by suspending the comminuted extrudates in a carrier liquid, if necessary with the addition of, other additives such as wetting agents, colorants, absorption-promoting substances, preservatives, antioxidants of light stabilizers.

Possible carrier liquids are homogeneous solvents or solvent mixtures, in which the extrudates in question do not dissolve. By way of example, physiologically compatible solvents such as water, alcohols such as ethanol, butanol, glycerine, propylene glycol, polyethylene glycols and mixtures thereof may be mentioned.

As wetting agents (dispersants), surfactants can be used. By way of example:

non-ionigenic surfactants, e.g. polyethoxylated castor oil, polyethoxylated/sorbitan monooleate, sorbitan monostearate, glycerine monostearate, polyoxyethyl stearate or alkylphenol polyglycol ethers;
ampholytic surfactants such as for example di-Na N-lauryl-β-iminodipropionate or lecithin;
anion-active surfactants, such as for example Na lauryl sulphate, fatty alcohol ether sulphates or mono/dialkylpolyglycol ether orthophosphate ester monoethanolamine salt; and
cation-active surfactants such as for example cetyltrimethylammonium chloride may be mentioned.

As further additives; for example:

Viscosity-raising and suspension-stabilizing substances such, as carboxymethylcellulose, methyl-cellulose and other cellulose and starch derivatives, polyacrylates, alginates, gelatine, gum Arabic, polyvinylpyrrolidone, polyvinyl alcohol, copolymers of methyl vinyl ether and maleic anhydride, polyethylene glycols, waxes, colloidal silicic acid or mixtures of the substances listed may be mentioned.

Semisolid preparations can be administered orally. They differ from the suspensions and emulsions described above only by their higher viscosity.

The active substances can also be used in combination with synergists or other active substances.

### EXAMPLES

Unless otherwise stated, the percentage values are weight percent based on the finished mixture.

### 1. Comparative Example

### Extrudate with Praziquantel

A powder mixture consisting of the active substance praziquantel (50% [w/w]) with a particle size d(0.9)=25 μm, see FIG. 1) and the additives Compritol® 888 ATO (49% [w/w]), a fatty base with the main component glycerol dibehenate (it also contains the mono- and triester, and smaller quantities of esters with C₁₆-C₂₀ fatty acids), and Aerosil® 200 (1% [w/w]), a pyrogenic colloidal silicon dioxide (also described as high disperse silicon dioxide), the use whereof contributes to the improvement of the flowability of the powder mass, was mixed in a laboratory mixer at room temperature (15 mins, 40 rpm) before the extrusion, and the powder-mixture was transferred to the gravimetric feed of the extruder.

For the melt extrusion, a constant speed twin screw extruder with a circular tool and blunt screw attachments was used.

In the course of the extrusion of this formula, many nozzle apertures blocked (the nozzle plate has a total of 67 nozzle holes each with a diameter of 0.3 mm), and as a result at constant revolution rate and feed rate the pressure rose continuously (FIG. 2). Owing to the pressure build-up behind the nozzle plate during the process with unmilled praziquantel, the exit rate of the extrudate from the nozzle apertures that remained clear increased very markedly.

### II. Example

### Extrudate with Finely Milled Praziquantel

The experiment of the comparative example was repeated using praziquantel which had been milled twice in an air jet mill (d(0.5)=1.7 μm, d(0.9)=3.6 μm, see FIG. 3).

The extrusion process with milled praziquantel with a drug substance loading of 50% and a nozzle diameter of 0.3 mm was uniform and reproducible at constant pressure. From the course of the process shown in FIG. 4 it can be seen that after about 12 minutes the pressure settled at a constant level and at 10 bar was markedly lower than in the process with unmilled praziquantel (FIG. 2), where the pressure had risen to almost 40 bar after 15 minutes' processing time Extrusion took place at uniform speed through all the nozzle apertures.

On comparison of the extrudate from comparative example I and this example, differences are seen: owing to the considerable friction, more lipid was pressed onto the surface of the extrudate, which can be very clearly seen under the scanning electron microscope; the surface of the extrudate is smooth and even. In contrast to this, the surface of the extrudate with milled praziquantel is uneven, the praziquantel particles are located directly under the surface and their shape is apparent through the layer.

### III. Comparative Example

### Extrudate with Unmilled Mesalazine

Extrudates with unwilled mesalazine (d(0.5)=10.7 μm, d(0.9)=44.0 μm, compare FIG. 5) were produced analogously to comparative example I. A powder mixture of 50% [w/w] unmilled mesalazine/49% [w/w] Compritol®/1% [w/w] high disperse silicon dioxide (Aerosil®) was used, and extrusion was performed through a nozzle plate with 0.3 mm diameter. Similar problems to those in comparative example I occurred in the production process. From the course of the process (FIG. 6) it is clear that, after 10 minutes the pressure settled at about 25 bar, but is still fluctuating really strongly. In this experiment also, partial blockage of the nozzle apertures was observed, but this did not occur so rapidly nor on such a large-scale as with praziquantel.

### IV. Example

### Extrudate with Milled Mesalazine

By milling twice in an air jet mill, mesalazine with a smaller particle size was obtained: d(0.5)=4.9 μm, d(0.9)=11.9 μm, see FIG. 7. After the milling, the particles exhibited a markedly altered shape.

Through the use of this milled mesalazine in the extrusion process analogously to comparative example III, it was possible to improve the process decisively. It can be seen in FIG. 8 that after 5 minutes the pressure had already settled at a constant level of 20 bar, and extrusion took place at uniform speed through all the nozzle apertures. Hence through the use of milled mesalazine the blockage of the nozzle apertures could be prevented, and the extrusion process thus improved.

On comparison of the extrudates, was found that the needle-shaped mesalazine crystals from the comparative example were largely destroyed during the extrusion, while the milled mesalazine powder from this example still had its original particle shape and size after the extrusion, which can for example clearly be seen under the optical microscope with a heatable slide. In contrast to this the needle-shaped praziquantel crystals were not destroyed during the extrusion and were also still present in the extrudate in their original shape and size.

On the basis of the results described above, the person skilled in the art would have, expected that the large needle-shaped mesalazine crystals would cause greater problems in the extrusion than the smaller praziquantel needles. However, the blockage of the nozzle apertures and the rapid pressure build-up caused by this did not occur so strongly with mesalazine as with praziquantel, because the large mesalazine needles were already largely comminuted by the shear, action of the extruder screws during the process.

### V. Comparative Example

### Extrudate with no Antistatic Agent

Extrudates were produced with milled praziquantel analogously to example II. A powder mixture of 50% [w/w] milled praziquantel/49% [w/w] Dynasan 116®/1% [w/w] high disperse silicon dioxide (Aerosil®) was used; Dynasan 116® is a fatty base, which 98% consists of glycerol tripalmitate. Extrusion was effected through a nozzle-plate with 0.3 mm diameter.

In the course of the extrusion of this formula, ever stronger electrical charges arose on the extrudates due to friction of lipophilic mass, on the nozzle inner surfaces, so that after a few minutes the extrudate was already strongly electrostatically attracted by the extruder itself and remained adhering to the nozzle head. Owing to this charging, some nozzle apertures also blocked and the pressure behind the nozzle plate increased, although milled praziquantel was used. The charges arising could be continuously recorded by means of the electrostatic sensor IZD 10-510 from SMC, which was attached directly before the nozzle plate during the process. During the extrusion in this comparative example; the electrostatic charge already reached a value of −5 kV after just 2 minutes (FIG. 9).

### VI. Example

### Extrudate with Antistatic Agent

Extrudates were produced analogously to comparative example V with the addition of 5 and 10% PEG 1500 as an antistatic agent. Powder mixtures of 50% [w/w] milled praziquantel/44% [w/w] Dynasan 116®/5% [w/w] PEG 1500/1% [w/w] Aerosil® or of 50% [w/w] milled praziquantel/39% [w/w] Dynasan 116®/10% [w/w] PEG 1500/1% [w/w] high disperse silicon dioxide (Aerosil®) were used. The cylinder temperature was about 60° C., which ensured melting of the PEG 1500 during the extrusion process.

In the course of the extrusion with 5% PEG, a few nozzle apertures again blocked, and after 3 minutes' process time electrostatic charges of 1-2 kV were measured before the nozzle plate. In contrast to this, the extrusion process with 10% proceeded uniformly and reproducibly with no blocked nozzle apertures and without measureable electrostatic charging(FIG. 9).

Through the addition of 10% PEG as an antistatic agent, it was possible to prevent electrostatic charging of the extrudate containing high purity glycerol triesters, and thus to improve the process, markedly.

### VII. Comparative Example

### Extrudate with Unmelted Antistatic Agent

Extrudates were produced analogously to example VI with the addition of 10% PEG 6000 as an antistatic agent. A powder mixture of 50% [w/w] milled praziquantel/39% [w/w] Dynasan 116®/10% [w/w] PEG 6000/1% [w/w] high disperse silicon dioxide (Aerosil®) was used. The cylinder temperature was at first about 60° C. and was then cooled after 8 minutes firstly to 55° C. and finally to 52° C. The melting range of PEG 6000 is about 55-60° C., so that after the lowering of the cylinder temperature the PEG was no longer present in melted form.

At first, analogously to example VI with 10% PEG 1500, the process took place uniformly and free from electrostatic charges, and all nozzle-apertures were clear (FIG. 10). After lowering of the temperature, the extrudates became electrostatically charged, some nozzle apertures blocked and as a result the pressure behind the nozzle plate increased.

It could be shown that the PEG should be present in melted form during the extrusion process in order to manifest its antistatic action.

### VIII. Comparative Example

### Extrudate with Unmilled Caffeine

Extrudates were produced analogously to comparative example I with unmilled caffeine. A powder mixture of 50% [w/w] unmilled caffeine (particle size d(0.9)=1170 μm)/49% [w/w] Compritol®/1% [w/w] high disperse silicon dioxide (Aerosil®) was used; extrusion was effected through a nozzle plate with 0.3 mm diameter. Similar problems arose in the production process to those in comparative example I. After 8 minutes' extrusion, the pressure had already increased so much through complete blockage of the nozzle apertures that the process had to be stopped.

### IX. Example

### Extrudate with Milled Caffeine

Caffeine with a smaller particle size was obtained by milling twice in an air jet mill.

By the use of this milled caffeine in the extrusion process analogously to example II, the process could be decisively improved. During the extrusion, after 3 minutes the pressure already settled at a constant level of 5 bar, and extrusion took place at uniform speed through all nozzle apertures. Hence through the use of milled caffeine it was possible to prevent blockage of the nozzle apertures and markedly improve the extrusion process.

The following extrudates were produced analogously to the previous examples:

### X. Example

| | |
|---|---|
| 70% [w/w] | praziquantel (milled, d(0.9) = 5.7 μm) |
| 17% [w/w] | glycerol tristearate (Dynasan ® 118) |
| 12% [w/w] | PEG 6000 |
| 1% [w/w] | high disperse silicon dioxide (Aerosil ®) |

Nozzle diameter: 0.3 mm (100% clear nozzle apertures during the extrusion), screw speed: 60 rpm, extrusion temperature: 67° C.

### XI. Example

| | |
|---|---|
| 50% [w/w] | praziquantel (milled, d(0.9) = 5.7 μm) |
| 29% [w/w] | glycerol tristearate (Dynasan ® 118) |
| 20% [w/w] | PEG 6000 |
| 1% [w/w] | high disperse silicon dioxide (Aerosil ®) |

Nozzle diameter: 0.2 mm (180 nozzle apertures, 100% clear nozzle apertures during the extrusion), screw speed: 60 rpm, extrusion temperature: 67° C.

### XII. Example

| | |
|---|---|
| 50% [w/w] | praziquantel (milled, d(0.9) = 5.7 μm) |
| 29% [w/w] | glycerol tristearate (Dynasan ® 118) |
| 20% [w/w] | PEG 6000 |
| 1% [w/w] | high disperse silicon dioxide (Aerosil ®) |

Nozzle diameter: 0.3 mm (100% clear nozzle apertures during the extrusion), screw speed: 60 rpm, extrusion temperature: 67° C.

### XIII. Example

| | |
|---|---|
| 50% [w/w] | praziquantel (milled, d(0.9) = 5.7 μm) |
| 49% [w/w] | glycerol tristearate (Dynasan ® 118) |
| 1% [w/w] | high disperse silicon dioxide (Aerosil ®) |

Nozzle diameter: 0.3 mm (3% clear nozzle apertures during the extrusion), screw speed: 60 rpm, extrusion temperature: 67° C.:

### Biological Example

### Acceptance Test in Cats

Forty cats were divided into 4 groups each of 10 animals. Extrudates from each of examples X to XIII were tested, each in one group of 10 cats, in the following way:

The quantity of extrudate which corresponds to a dosage of 5 mg praziquantel per kg body, weight was added to dry cat food as top dressing, and offered to the cats at the normal feeding times. All the cats ate all of the food. This was regarded as evidence of 100% acceptance.

After a one-week pause, the experiment was repeated, during which however tinned food (moist) for cats was used. The same result was observed as in the test with dry food, and assessed as 100% acceptance.

### FIGURES

FIG. 1: Particle size distribution of unmilled praziquantel, d(0.5)=6.2 μm, d(0.9)=25.1 μm, measured by laser diffractometry after wet dispersion (Mastersizer 2000 Ver. 5.54, Malvern Instruments, Malvern UK).

FIG. 2: Course of the extrusion process with 50% unmilled praziquantel/49% Compritol®/1% high disperse silicon dioxide (Aerosil®), nozzle plate 0.3 mm diameter.

FIG. 3: Particle size distribution of twice milled praziquantel, d(0.5)=1.7 μm, d(0.9)=3.6 μm, measured by laser diffractometry after wet dispersion (Mastersizer 2000 Ver. 5.54; Malvern Instruments, Malvern UK).

FIG. 4: Course Of the extrusion process with 50% milled praziquantel/49% Compritol®/1% high disperse silicon dioxide (Aerosil®), nozzle plate 0.3 mm diameter.

FIG. 5: Particle size distribution of unmilled mesalazine, d(0.5)=10.7 μm, d(0.9)=44.0 μm, measured by laser diffractometry after wet dispersion (Mastersizer 2000 Ver. 5.54, Malvern Instruments, Malvern UK).

FIG. 6: Course of the extrusion process with 50% unmilled mesalazine/49% Compritol®/1% high disperse silicon dioxide (Aerosil®), nozzle plate 0.3 mm diameter.

FIG. 7: Particle size distributor of twice Milled mesalazine, d(0.5)=4.9 μm, d(0.9)=11.9 μm, measured by laser diffractometry after wet dispersion (Mastersizer 2000 Ver. 5.54, Malvern Instruments, Malvern UK).

FIG. 8: Course of the extrusion process with 50% milled mesalazine/49% Compritol®/1% high disperse silicon dioxide (Aerosil®), nozzle plate 0.3 mm diameter.

FIG. 9: Electrostatic charge before the nozzle plate during the extrusion process with 0, 5, and 10% PEG 1500 content respectively.

FIG. 10: Course of the extrusion process with 50% milled praziquantel/39% Dynasan 116®/10% PEG 6000/1% high disperse silicon dioxide (Aerosil®).

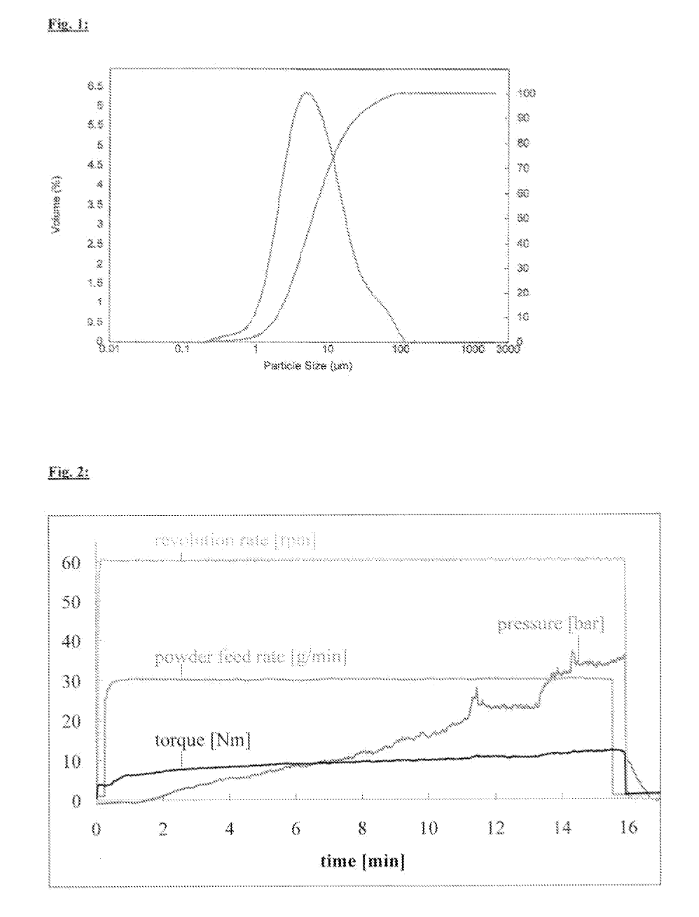

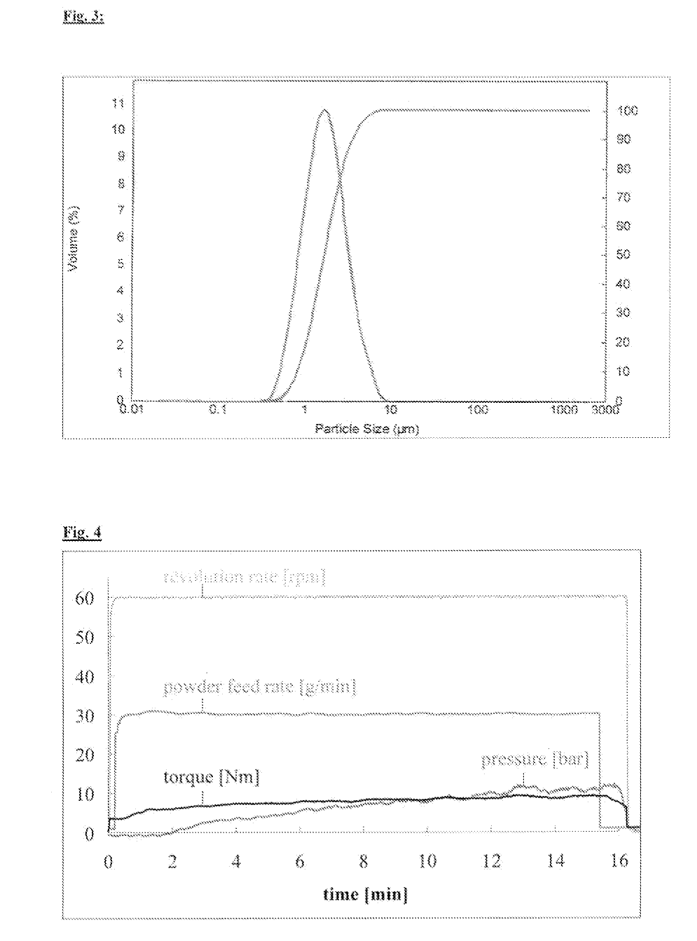

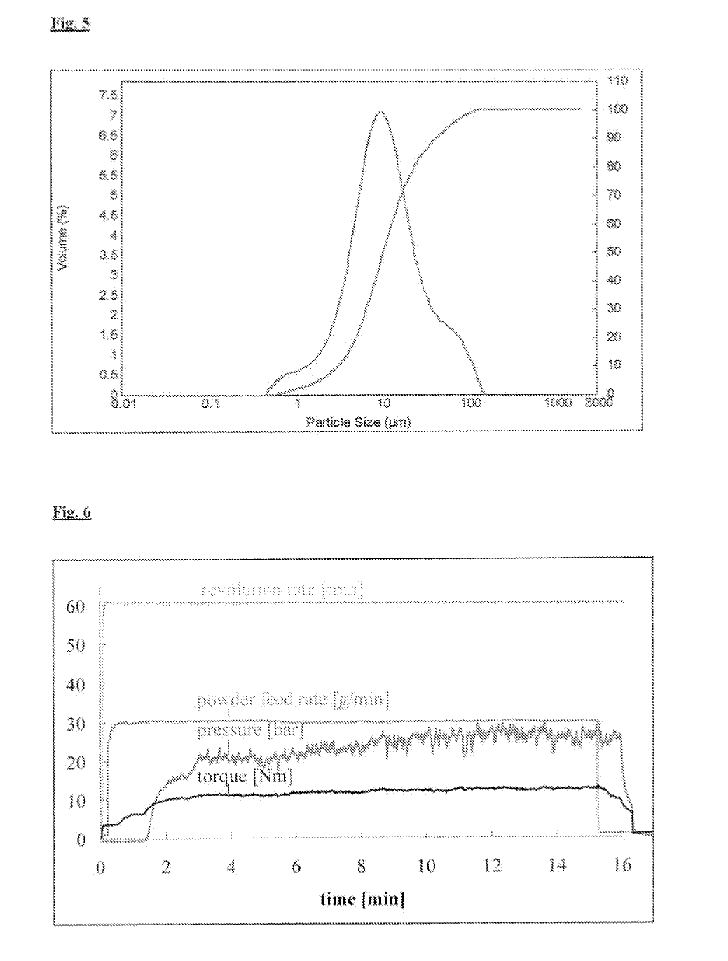

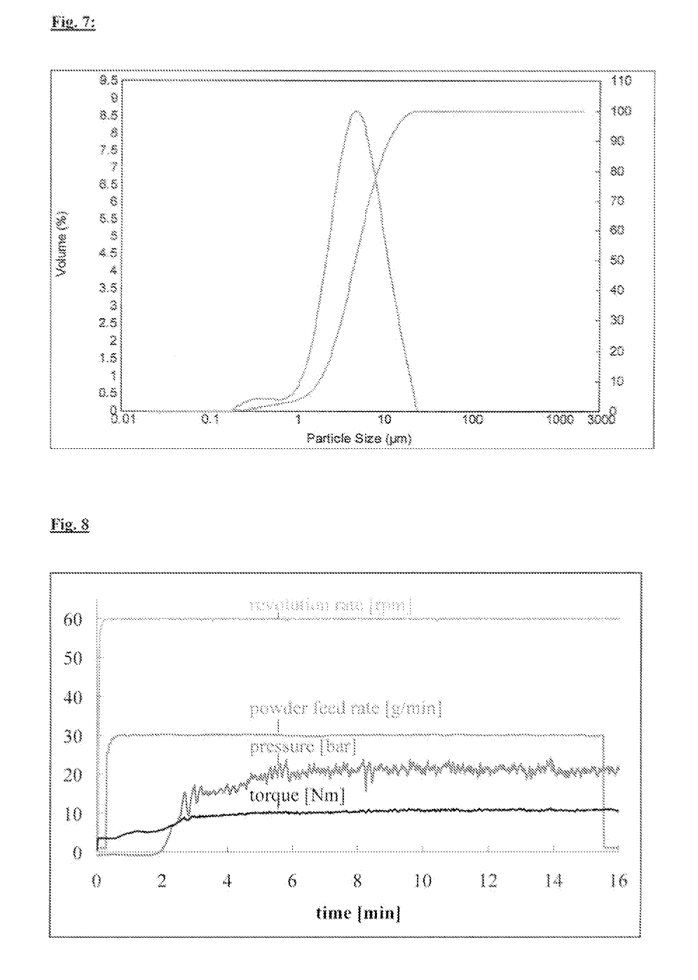

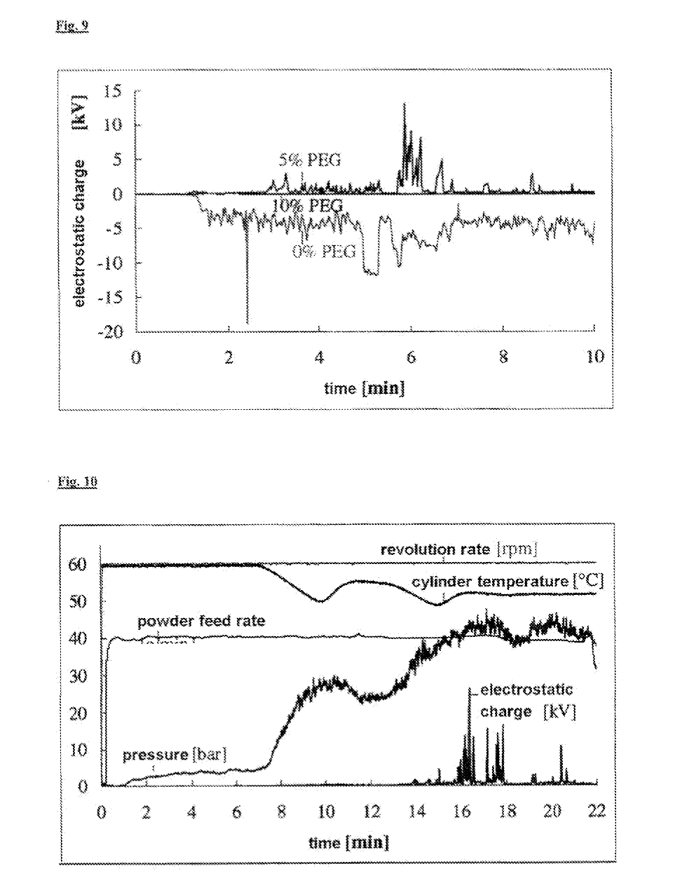

### LITERATURE

Adeyeye C. M., Price J. C., 1991. Development and Evaluation of Sustained-Release Ibuprofen-Wax Microspheres. 1. Effect of Formulation Variables an Physical Characteristics. Pharm. Res. 8, 1377-1383.
Adeyeye C. M., Price J. C., 1994. Development and Evaluation of Sustained-Release Ibuprofen-Wax Microspheres. 2. In-Vitro Dissolution. Pharm. Res. 11, 575-579.
Ahrens G., Mentrup E., Maas J., Radau M., 1998. Production of taste-masked preparations of antibacterially active quinolone derivatives. EP 1998/0855183.
Alderborn G., Nyström C., 1982. Studies on direct compression of tablets. 3. The effect on tablet strength of changes in particle-shape and texture obtained by milling. Acta Pharm. Suec. 19, 147-156.

Al-Omran M. F., Al-Suwayeh S. A., El-Helw A. M., Saleh S. I., 2002. Taste masking of diclofenac sodium using microencapsulation. J. Microencapsul. 19, 45-52.
Barra J., Lescure F., Doelker E., 1999. Taste masking as a consequence of the organisation of powder mixes. Pharm. Acta Helv. 74, 37-42.
Barthelemy P., Laforet J. P., Farah N., Joachim J., 1999. Compritol® 888 ATO an innovative hot-melt coating agent for prolonged-release drug formulations. Eur. J. Pharm. Biopharm. 47, 87-90.
Bienz M., 1996. Taste masking strategies for drug dosage forms. Manufacturing Chemist. 67, 17-20.
Breitkreutz J., El-Saleh F., Kiera C., Kleinebudde P., Wiedey W., 2003. Pediatric drug formulations of sodium benzoate: II. Coated granules with a lipophilic binder. Eur. J. Pharm. Biopharm. 56, 255-260.
Cabrera F. A., 2002. Solid Phase Dispersion of Quinolone-OR Naphthyridonecarboxylic acids. WO 2002/058669.
Cerca M., Zheng W. J., Young C. R., McGinity J. W., 2004. A novel powder coating process for attaining taste masking and moisture protective films applied to tablets. Int. J. Pharm. 279, 127-139.

Chun M. K., Choi H. K., 2004. Preparation and characterization of enrofloxacin/carbopol complex in aqueous solution. Arch. Pharm. Res. 27, 670-675.

Farah N., Barthelemy P., Joachim J., 1998. Method for preparing a pharmaceutical composition with modified release of the active principle, comprising a matrix. WO 1998/14176.

Huber G. R., Jones D. R., Kuenzi J. C., Kuenzi K. D., Cabrera F. A, 2003: Animal Feeds including Actives and Methods of using same. WO 2003/030653.

Jbilou M., Ettabia A., Guyot-Hermann A. M., Guyot J. C., 1999. Ibuprofen Agglomerates Preparation by Phase Separation. Drug Dev. Ind. Pharm. 25, 297-305.

Kalbe J., Hopkins T., 1998. Orally administrable granules of hexahydropyrazine derivatives. WO 1908/03157.

Katsuragi Y., Mitsui Y., Umeda T., Otsuji K., Yamasawa S., Kurihara K., 1997. Basic studies for the practical use of bitterness inhibitors: Selective inhibition of bitterness by phospholipids. Pharm. Res. 14, 720-724.
Kim E.-H., Choi H. K., 2004. Preparation of various solid-lipid beads for Drug Delivery of Enrofloxacin. Drug Deliv. 11, 365-370.

Li F.-Q., Hu J.-H., Deng J.-X., Su H., Xu S., Liu J.-Y., 2006. In vitro controlled release of sodium ferulate from Compritol® 888 ATO-based matrix tablets. Int. J. Pharm. 324, 152-157.
Li S. P., Martellucci S. A., Bruce R. D., Kinyon A. C., Hay M. B., Higgins J. D., 2002. Evaluation of the film-coating properties of a hydroxyethyl cellulose/hydroxypropyl methylcellulose polymer system. Drug Dev. Ind. Pharm. 28, 389-401.
Lovrecich M., Nobile F., Rubessa F., Zingone G., 1996. Effect of ageing on the release of indomethacin from solid dispersions with Eudragits. Int. J. Pharm. 131, 247-255.
Lu M. Y. F., Borodkin S., Woodward L., Li. P., Diesner C., Hernandez L., Vadnere M., 1991. A Polymer Carrier System for Taste Masking of Macrolide Antibiotics. Pharm. Res. 8; 706-712.
Moisescu C., Jana C. Habelitz S., Carl G., Rüssel C., 1999. Oriented fluoroapatite glass-ceramics. J. Nonery. Sol. 248, 176-182.
Mirghani A., Idkaidek N. M., Salem M. S., Najib N. M., 2000. Formulation and release behavior of diclofenac sodium in Compritol® 888 ATO matrix beads encapsulated alginate. Drug Dev. Ind. Pharm. 26, 791-795.
Nabil, F. et al., 1998. Method for preparing a pharmaceutical composition with modified release of the active principle, comprising a matrix method or preparing a pharmaceutical composition with modified release of the active principle, comprising a matrix. FR2753904 (A1).
Ohta M., Buckton G., 2004. The use of inverse gas chromatography to assess the acid-base contributions to surface energies of cefditoren pivoxil and methacrylate copolymers and possible links to instability. Int. J. Pharm. 272, 121-128.
Pearnchob N., Dashevsky A., Siepmann J., Bodmeier R, 2003a. Shellac used as coating material for solid pharmaceutical dosage forms: understanding the effects of formulation and processing variables. Stp Pharma Sciences. 13, 387-396.
Pearnchob N., Siepmann J., Bodmeier R., 2003b. Pharmaceutical applications of shellac: Moisture protective and taste masking coatings and extended-release matrix tablets. Drug Dev. Ind. Pharm. 29, 925-938.
Petereit H. U., Weisbrod W., 1999. Formulation and process considerations affecting the stability of solid dosage forms formulated with methacrylate copolymers. Eur. J. Pharm. Biopharm. 47, 15-25.
Petereit H. U. Meier C., Gryczke A., 2003. Melt extrusion of active substance salts. WO 2003/072083.
Petereit H. U., Meier C., Gryczke A., 2004. Process for the production of an oral drug form with direct disintegration and active substance-release. WO 2904/060976.
Prompruk K., Govender T., Zhang S., Xiong C. D., Stolnik S., 2005. Synthesis of a novel PEG-blockpoly (aspartic acid-stat-phenylalanine) copolymer shows potential for formation a micellar drug carrier. Int. J. Pharm. 297, 242-253.
Rasenack N., Müller B. W., 2002. Crystal habit and tableting behaviour. Int. J. Pharm., 244, 45-57.
Reitz C., Kleinebudde P., 2007. Solid lipid extrusion or sustained, release dosage forms. Eur. J. Pharm. Biopharm. 67, 440-448.
Schubert M. A., Schicke B. C., Müller-Goymann C. C., 2005. Thermal analysis of the crystallization and melting behavior of lipid matrices and lipid nanoparticles containing high amounts of lecithin. Int. J. Pharm. 298, 242-254.
Shirai Y., Sogo K., Yamamoto K., Kojima Fujioka H., Makita H., Nakamura Y., 1993. A Novel Fine Granule System for Masking Bitter Taste. Biol. Pharm. Bull. 16, 172-177.
Sohi H., Sultana Y., Khar R. K., 2004. Taste masking technologies, in oral pharmaceuticals: Recent developments and approaches. Drug Dev. Ind. Pharm. 30, 429-448.
Suzuki. H., Onishi Takahashi Y., Iwata Machida Y., 2003. Development of oral acetaminophen chewable tablets WITH inhibited bitter taste. Int. J. Pharm 251, 123-132.
Suzuki H., Onishi H., Hisamatsu S., Masuda K., Takahashi Y. Iwata M., Machida Y., 2004. Acetaminophen-containing chewable tablets with suppressed bitterness and improved oral feeling. Int. J. Pharm. 278, 51-61.
Takagi S. Toko K., Wada K., Ohki T., 2001. Quantification of suppression of bitterness using an electronic tongue. J. Pharm. Sci. 90, 2042-2048.
Thombre A. G., 2004. Palatable controlled-release formulations for companion animals. WO 2004/014346.
Wang H., Zhang R., 1995. Compaction behaviour of paracetamol powders of different crystal shapes. Drug Dev. Ind. Pharm. 21; 803-868.
Windbergs M., Strachan C. J., Kleinebudde P., 2008 Understanding the solid-state behaviour of triglyceride lipid extrudates and its influence on dissolution. Eur. J. Pharm. Biopharm. In Press.
Wong L. W., Pilpel N., 1990. The effect of particle shape on the mechanical properties of powders. Int. J. Pharm. 59, 145-154.
Yue Y., Moisescu C., Carl G., Rüssel C., 1999. Influence of suspended iso- and anisometric crystals on the flow behaviour of fluoroapatite glass melts during extrusion. Physics and Chemistry of Glasses. 40, 243-247.
Zhou F., Vervaet C., Remon J. P., 1996. Matrix pellets based on the combination of waxes, starches and maltodextrins. Int. J. Pharm. 133, 155-160.
Zhou F., Vervaet C., Schelkens M., Lefebvre R., Remon J. P., 1998. Bioavailability of ibuprofen from matrix pellets based on the combination of waxes, and starch derivatives. Int. J. Pharm. 168, 79-84.

1. An extrudate containing at least one pharmaceutically active substance in the form of needles,
characterized in that the ratio of the particle size of the needle-shaped pharmaceutically active substance to the strand diameter is at least 1:15.

2. The extrudate according to claim 1, wherein the ratio of the particle size of the needle-shaped pharmaceutically active substance to the strand diameter is at least 1:20.

3. The extrudate according to claim 1, wherein the ratio of the particle size of the needle-shaped pharmaceutically active substance to the strand diameter is at least 1:25.

4. The extrudate according to claim 1, wherein the strand diameter is 0.5 mm or less.

5. The extradate according to claim 1, wherein the extrudate contains a lipid base as an additive.

6. The extrudate according to claim 5, wherein the lipid base is a glycerol ester with C12-C24 fatty acids.

7. The extrudate according to claim 5, wherein the lipid base is a glycerol diester.

8. The extrudate according to claim 5, wherein the lipid base is glycerol dibehenate.

9. The extrudate according to claim 5, wherein the lipid base is a glycerol triester.

10. The extrudate according to claim 9, wherein the glycerol triester is selected from the group consisting of glycerol trimyristate, glycerol tripalmitate or, glycerol tristearate.

11. The extrudates according to claim 10, wherein the glycerol triester is glycerol tristearate.

12. The extrudates according to claim 1, further comprising an antistatic agent.

13. The extrudates according to claim 1, wherein the extrudate was extruded below the lower limit of the melting range of the base contained therein.

14. (canceled)

15. A medicament comprising an extrudate according to claim 1 and one or more pharmaceutically acceptable auxiliary substances and/or additives.

16. The extrudate of claim 12, wherein the antistatic agent is polyethylene glycol.
